(19) United States
(12) Patent Application Publication
(10) Pub. No.: US 2009/0197286 A1
(43) Pub. Date: Aug. 6, 2009

(54) Methods and Kit for Diagnosing T1DM

(75) Inventors: Nathan Karin, Haifa (IL); Naim Shehadeh, Kfar-Yassif (IL); Gizi Wildbaum, Kiryat Yam (IL)

Correspondence Address: MARTIN D. MOYNIHAN d/b/a PRTSI, INC., P.O. BOX 16446, ARLINGTON, VA, 22215, US

(73) Assignees: RAPPAPORT FAMILY INSTITUTE FOR RESEARCH IN THE MED, Haifa (IL); RAMBAM MEDICAL CENTER RESEARCH FUND, Haifa (IL)

(21) Appl. No.: 12/223,615

(22) PCT Filed: Feb. 6, 2007
(86) PCT No.: PCT/IL2007/000156

§ 371 Date: Jan. 2, 2009

Related U.S. Application Data
(60) Provisional application No. 60/765,220, filed on Feb. 6, 2006.

(51) Int. Cl. G01N 33/53; G01N 33/564

(52) U.S. Cl. 435/7.92; 436/506

(57) ABSTRACT

A method of diagnosing Type 1 Diabetes Mellitus (T1DM) in a subject in need thereof is provided. The method comprising determining a presence and/or a level of antibodies in a biological sample of the subject, wherein the presence or level above a predetermined threshold is indicative of T1DM, thereby diagnosing T1DM in the subject. Also provided are a kit for diagnosing T1DM and a method of monitoring anti diabetic treatment.

### FIELD AND BACKGROUND OF THE INVENTION

The present invention relates to a novel method and kit for diagnosing T1DM.

Diabetes mellitus is a group of chronic metabolic diseases characterized by high blood sugar (glucose) levels, which result from defects in insulin secretion, or action, or both. If left untreated, Diabetes mellitus can cause serious health complications including renal failure, heart disease, stroke, and blindness. Approximately 15 million Americans (about 8% of the population) have diabetes. From an economic perspective, the total annual economic cost of diabetes in 1997 was estimated to be 98 billion dollars in the United States.

Type-1 diabetes mellitus (T1DM), also called juvenile diabetes, or insulin-dependent diabetes begins in childhood or adolescence, and is most commonly found at 8-12 years of age. In the United States, approximately 5 to 10 percent of diagnosed diabetes is T1DM.

T1DM is the result of organ-specific autoimmune destruction of the insulin-secreting β-cells in the pancreatic islets of Langerhans, which leads to no production of insulin by the body. It has become evident that T1DM is a multifactorial autoimmune disease mediated by T cells in which both CD4⁺ and CD8⁺ T cells and macrophages are required for β-cell destruction. Proinflammatory mediators and adhesion molecules are involved in directing the invasion and accumulation of these self reactive leukocytes into the pancreatic islets of Langerhans.

A specific subgroup of T1DM is referred to Latent Autoimmune Diabetes in Adults [LADA; also called Late-onset Autoimmune Diabetes of Adulthood, “Slow Onset Type 1” diabetes, or “Type 1.5 (Type one-and-a-half)” diabetes]. This medical condition is associated with islet autoantibodies similar to type I diabetes but due to its late onset is often misdiagnosed as type 2 diabetes milletus (T2DM).

In early stages LADA, typically presents as non-obese Type 2 diabetes mellitus phenotypes (patient is thin or of normal weight). Unfortunately, in its physiological display, LADA more closely resembles juvenile (Type 1) diabetes and shares common characteristics for metabolic dysfunction, genetics, and autoimmune features with the insulin dependent disease.

Current diagnosis of T1DM is based on detecting autoantibodies to glutamic acid decarboxylase (GAD), islet cell antigen (ICA) 512/IA-2 and insulin. Unfortunately these tests are not sensitive enough to cover all T1DM patients. At the onset of disease using anti-GAD, IA-2A and anti-Insulin (IAA) antibodies in combination offers >85% sensitivity. The sensitivity of anti-GAD alone is 70-80%, IA-2A 50-70% and anti-Insulin (IAA) 30-50%, with the variances in the ranges reflecting the population differences between studies (Clive et al., Autoimmunity reviews, 5:424-428, 2006; Polly et al., Diabetes Care, 24:398, 2001); therefore the present tests do not offer sufficient diagnostic accuracy and reliability.

Moreover, after the diagnosis of diabetes, patients do not display a notable antibody titer to insulin or ICA 512, and only a limited number of them display an antibody titer to GAD. The diagnosis of autoimmune diabetes in adults is therefore not easy and is usually considered after the failure of treating the patients with oral hypoglycemic medication (several years after the diagnosis of diabetes). In this case only a small percentage of patients will have a positive titer of anti-GAD antibodies.

As mentioned above, LADA patients are often mistakenly diagnosed with Type 2 Diabetes based on their age at the time of diagnosis and currently available diagnosis tools including auto antibody production and C-peptide level (which gives an indication of the level of insulin produced by the pancreas). Such misdiagnosis often leads to wrong treatment regimen (sulfonylureas or other diabetes pills instead of oral insulin at onset). It should be noted that although LADA patients may respond to oral diabetes medications and lifestyle changes, their beta cells continue to be destroyed and LADA patients should be closely monitored. It is therefore of great importance to be able to diagnose type I diabetes (including LADA) correctly in order to administer the right treatment.

Chemokines are proinflammatory proteins which mediate autoimmune diseases. This role has made these proteins valid targets for therapy. The chemokines CCL2 (MCP-1), CCL3 (MIP-1α) and CCL4 (MIP-1β), were found to be expressed in the inflamed pancreas during T1DM in mouse experimental models [Cameron, M. J. et al., J Immunol 165:1102 (2000)].

In addition, CCL3 and CCL4 were found to be up-regulated in individuals in risk of T1DM development (family history), that exhibited high levels of T1DM specific autoantibodies (ICA, GAD and IA-2 autoantibodies) [Hanifi-Moghaddam, et al., Diabetic Medicine 231: 56 (2006)]. Importantly, the presence of autoantibodies for CCL3, was not determined in the foregoing studies, nor was it suggested as a potential diagnostic tool.

There is thus a widely recognized need for, and it would be highly advantageous to have methods and kits for diagnosing T1 DM diabetes which are devoid of the above limitations.

### SUMMARY OF THE INVENTION

According to one aspect of the present invention there is provided a method of diagnosing Type 1 Diabetes Mellitus (T1DM) in a subject in need thereof, the method comprising determining a presence and/or a level of anti-CCL3 antibodies in a biological sample of the subject, wherein the presence or level above a predetermined threshold is indicative of T1DM, thereby diagnosing T1DM in the subject.

According to another aspect of the present invention there is provided a kit for diagnosing T1DM, the kit comprising a packaging material and at least one reagent for determining anti-CCL3 antibodies in a biological sample of the subject.

According to yet another aspect of the present invention there is provided a method of monitoring anti diabetic treatment in a subject in need thereof, the method comprising exposing the subject to an anti-diabetic treatment; and determining a presence and/or a level of anti-CCL3 antibodies in a biological sample of the subject, wherein an alteration in a level of the anti-CCL3 antibodies in the biological sample following exposing the subject to the anti-diabetic treatment is indicative of treatment efficacy.

According to further features in preferred embodiments of the invention described below, determining a presence and/or a level of anti-CCL3 antibodies is effected following and optionally prior to the step exposing the subject to an anti-diabetic treatment.

According to still further features in the described preferred embodiments the anti-diabetic treatment comprises a drug selected from the group consisting of insulin, glucagon, glucose, biguanide, chromium, ginseng, magnesium and vanadium.

According to still further features in the described preferred embodiments the anti diabetic treatment is selected from the group consisting of pancreas transplantation, islet cell transplantation, and a life-style regimen.

According to still further features in the described preferred embodiments determining a presence or a level of anti CCL3 antibodies is effected ex vivo.

According to still further features in the described preferred embodiments the T1DM comprises Latent Autoimmune Diabetes in Adults (LADA).

According to still further features in the described preferred embodiments determining a presence or a level of anti CCL3 antibodies is effected prior to the elicitation of an autoantibody specific to a T1DM disease state.

According to still further features in the described preferred embodiments the method comprises determining a presence or a level of an autoantibody specific to a T1DM disease state.

According to still further features in the described preferred embodiments the kit further comprises at least one reagent for determining a presence or level of an autoantibody specific to a T1DM disease state.

According to still further features in the described preferred embodiments the autoantibody specific for a T1DM disease state is specific for an antigen selected from the group consisting of glutamic acid decarboxylase (GAD), islet cell antigen (ICA) 512/IA-2 and insulin.

According to still further features in the described preferred embodiments the threshold comprises an anti-CCL3 titer of log₂Ab 10-15.

According to still further features in the described preferred embodiments the kit comprises instructions as follows: wherein an anti-CCL3 titer of log₂Ab 10-15 is indicative of T1 DM.

According to still further features in the described preferred embodiments determining a presence and/or a level of anti-CCL3 antibodies is effected by ELISA.

The present invention successfully addresses the shortcomings of the presently known configurations by providing a novel method and kit for diagnosing T1DM.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a bar graph depicting autoantibody titers in 10 subjects with newly diagnosed with T1DM (within one week of diagnosis). For every patient, bars depict, from left to right, the autoantibody titers for IP-10, CXCL8 (IL-8), CCL2 (MCP-1), CCL3 (MIP-1α) and CCL4 (MCP-1β). Note the high and selective autoantibody response to CCL3 in 9 out of 10 subjects.

FIG. 2 is a bar graph depicting the percentage of subjects showing a positive CCL3 autoantibody titer in subjects with new onset diabetes (n=30), prediabetic subjects (n=20), prolonged T1DM (n—38) and normal controls (n=20).

FIG. 3 is a bar graph depicting the percentage of subjects showing a positive autoantibody titer for the T1DM related autoantibodies: Anti-Insulin (CIAA), Anti-ICA, anti-GAD, a combination of all three diagnostic markers and Anti-CCL3, in 30 subjects with new onset diabetes.

### DESCRIPTION OF THE PREFERRED EMBODIMENTS

The present invention is of a method and kit for diagnosing T1DM.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Type 1 diabetes mellitus (T1DM) is the result of organ-specific autoimmune destruction of insulin-secreting β-cells in the pancreatic islets of Langerhans, which eventually leads to no production of insulin by the body. α-cell destruction starts years before the appearance of disease symptoms. It has been estimated that only 10% of the total α-cell mass remains at the time of clinical onset. Therefore, when the disease is finally diagnosed, critical α-cell depletion and insulin dependency may have already occurred.

A Latent Autoimmune Diabetes in Adults (LADA) is a subgroup of T1DM, which is often misdiagnosed as non-obese Type 2 diabetes mellitus due to its late onset, which is characteristic of Type 2 diabetes mellitus (T2DM). LADA patients typically present with more preserved beta-cell function than those with classic T1DM but usually experience marked loss of beta-cell function within 3 years of diagnosis, which eventually results in insulin dependency. Although LADA patients may respond to treatment against T2DM (oral hypoglycemic diabetes medications and lifestyle changes), their β cells continue to be destroyed, and thus treatment often requires rapid escalation of the oral treatment and early use of insulin. However, continued β cell destruction exposes these patients to a deterioration in health which can lead to a life threatening condition. The use of a screening tool for identifying LADA early will improve glycemic control and prevent undesired complications.

If left untreated, Diabetes mellitus can cause serious health complications including renal failure, heart disease, stroke, and blindness. Therefore, methods for accurate and early identification of T1DM are needed so as to diagnose T1DM before symptom manifestation and disease progression.

Current antibody-based methods of diagnosing T1DM (e.g., GAD, ICA 512/IA-2 and insulin) are not sensitive enough to cover all T1DM patients.

CCL3 is a chemokine that was found together with other chemokines to be expressed in the inflamed pancreas during T1DM in mouse experimental models [Cameron, M. J. et al., J Immunol 165:1102 (2000)]. Similar results were obtained in human studies [Hanifi-Moghaddam, et al., Diabetic Medicine 231: 56 (2006)].

While reducing the present invention to practice the present inventors uncovered elicitation of anti-CCL3 autoantibodies in T1DM-diagnosed subjects in general and -subjects with more than 5 years duration of T1DM in particular.

As is illustrated hereinbelow in the Examples section which follows, the present inventors have shown that early diagnosed T1DM subjects developed an exclusive autoantibody response to CCL3, and not to other inflammatory mediators (Example 1 of the Examples section). The present inventors have also shown that anti-CCL3 autoantibodies were present in 90% of the subjects (Example 2 of the Examples section), which is the same percentage as was found for the combined presentation of the conventionally used autoantibodies (anti-ICA, GAD, and CIAA autoantibodies—where 93% of the subjects tested were positive). In addition, the present inventors have shown that anti-CCL3 autoantibodies are present in high percentages in prediabetics (e.g., first degree relatives of patients with T1DM with positive autoantibodies) as well as in subjects that are within 5 years or more since diagnosis (see Example 2 of the Examples section).

Altogether, the present findings can be harnessed for the diagnosis of T1DM well before the onset of severe disease symptoms, and its sensitivity is such, that it can replace the combined sensitivity of all three-antibody based detection assays which are currently in use.

Thus, according to one aspect of the present invention, there is provided a method of diagnosing Type 1 Diabetes Milletus (T1DM) in a subject in need thereof. The method comprising determining a presence or a level of anti-CCL3 antibodies in a biological sample of the subject, wherein the presence or level above a predetermined threshold is indicative of T1DM, thereby diagnosing T1DM in the subject.

As used herein the term “T1DM” refers to a diabetes which is characterized by decreases in, or the complete absence of, the production of insulin. T1DM is also referred to as “childhood,” “juvenile,” or “insulin-dependent” diabetes. The pathophysiology may be autoimmune mediated or autoimmune independent. The latter may include exposure to vitamin D3, chemicals and drugs that specifically destroy pancreatic cells (e.g., N-3-pyridylmethyl-N′-p-pnitrophenyl urea and streptozotocin) and other pancreatic problems including, trauma, pancreatitis and tumors. T1DM according to the present invention can exhibit onset in early childhood and in adults (e.g., individuals over 25 years of age). The disease can be manifested with a non-insulin-dependent stage. Thus, T1DM according to the present invention also includes Latent Autoimmune Diabetes in Adults (LADA) and maturity onset diabetes in the young (MODY).

As used herein the phrase “subject in need thereof” refers to a mammal preferably a human subject who is at risk of T1DM [e.g., a genetically predisposed subject, a subject with medical and/or family history of T1DM, a subject who has been exposed to T1DM inducing drugs, chemicals or pathogens) and/or a subject who exhibits suspicious clinical signs of T1DM (e.g., polyurea, poloydipsia, weight loss and blood sugar levels of over 200 mg/dl or ≧126 mg/dl after 8 hour fasting). The subject may or may not have received anti-diabetic drugs. Additionally or alternatively, the subject in need thereof can be a healthy human subject undergoing a routine check up.

As used herein the term “diagnosing” refers to classifying a disease or a symptom as an inflammatory disease, determining a severity of such a disease, monitoring disease progression, forecasting an outcome of a disease and/or prospects of recovery.

As mentioned hereinabove, the method of this aspect of the present invention is effected by determining a presence or a level of anti-CCL3 antibodies in a biological sample obtained from the subject.

As used herein the term “CCL3” (also referred to herein as Macrophage inflammatory protein-1α(MIP-1α)] refers to the C—C chemokine of the GenBank Accession No. NP_{—}002974, that is typically involved in the acute inflammatory state in the recruitment and activation of polymorphonuclear leukocytes.

As used herein the phrase “anti-CCL3 antibodies” refers to any autoantibodies (self) which bind any epitope of CCL3. Autoantibodies of the present invention can be of any class [e.g., IgG (subclass 1-4), IgM, IgA (subclass 1-2), IgD, and IgE)). Of note, an IgG antibody population over IgM was found to associate with disease progression in mice [Hutchings et al., Diabetes, 46, 5: 779-784 (1997)]. Also, it was found that the highest risks for progression to type 1 diabetes is associated with high-titer IA-2A and IAA, IgG2, IgG3, and/or IgG4 subclass, and antibodies to the IA-2-related molecule IA-2beta [Achenbach et al., DIABETES, 53: 384-92 (2004)].

According to one embodiment of the present invention autoantibodies of the present invention can be detected in ex-vivo biological sample (removed from the subject) or by in vivo detection.

As used herein the phrase “biological sample” refers to an antibody-containing sample of cell, tissue or fluid derived from the subject. Antibodies present in the sample are typically found within cytoplasmic membrane-bound compartments (e.g., endoplasmic reticulum and Golgi apparatus) and on the surface of B lymphocytes (which synthesize antibody molecules) and immune effector cells such as, mononuclear phagocytes, natural killer (NK) cells and mast cells, which express specific receptors for binding antibody molecules. Antibodies are also present in the plasma (i.e., fluid portion) of the blood and in the interstitial fluid of the tissues. Antibodies can also be found in secretory fluids such as mucus, synovial fluid, sperm and milk into which certain types of antibody molecules are specifically transported.

General examples of antibody containing biological samples include, but are not limited to, tissue sample such as a pancreatic tissue, digestive tissue and/or a biological fluid such as blood, serum, plasma, lymph, bile fluid, urine, saliva, sputum, synovial fluid, semen, tears, cerebrospinal fluid, bronchioalveolar large fluid, ascites fluid and pus.

Procedures for obtaining biological samples (i.e., biopsying) from subjects are well known in the art. Such procedures include, but are not limited to, blood sampling, joint fluid biopsy, cerebrospinal biopsy and lymph node biopsy. These and other procedures for obtaining tissue or fluid biopsies are described in details in http://www.healthatoz.com/healthatoz/Atoz/search.asp.

Preferably, in diabetic subjects, samples are taken before the start or within 7 days of insulin treatment. When possible, biological samples such as serum may be transferred to aliquots and stored frozen at −80° C. until further analysis.

As mentioned hereinabove, the method of this aspect of the present invention is effected by determining a presence or a level of anti-CCL3 antibodies in a biological sample of the subject, wherein a presence of level of ant-CCL3 antibodies above a predetermined threshold (i.e., the level of the same in a biological sample obtained from a healthy individual) is indicative of T1DM.

The particular assay protocol chosen for detecting the presence or level of anti-CCL3 antibodies is not critical, and it is necessary only that the assay be sufficiently sensitive to detect the above mentioned predetermined threshold level of the autoantibodies. It will be appreciated that in the very early stages of β-cell destruction, very low levels of autoantibodies may be present. Thus, the presence of any autoantibodies above the negative background or control level (e.g., of a healthy sample) will be diagnostic of the prediabetic condition.

According to a preferred embodiment of the present invention, a predermined threshold of comprising an anti-CCL3 log₂Ab titer of 10 to 15 is indicative of T1DM.

Regardless of the procedure employed, once the biological sample is obtained, the titer (number) of antibody molecules for CCL3 in the biological sample is determined.

Antibody titer can determined by techniques which are well known in the art such as ELISA (direct or indirect) and dot blot using an immobilized antigen (see for Example Abbas, Lichtman and Pober “Cellular and Molecular Immunology”. W.B. Saunders International Edition 1994 pages 56-59). Specifically, the antigen (i.e., any CCL3 epitope or mimetics thereof) is preferably immobilized on a solid support. To avoid non-specific binding of antibodies, the solid support is preferably coated with a nonantigenic protein as well. A peptide is typically immobilized on a solid support (matrix) by adsorption from an aqueous medium, although other modes of immobilization applicable to proteins and peptides well known to those skilled in the art can be used.

As used herein the phrase “solid support” refers to a non-aqueous matrix to which a reagent of interest (e.g., CCL3 epitope) can adhere. Examples of solid supports, include, but are not limited to, solid supports formed partially or entirely of glass (e.g., controlled pore glass), polysaccharides (e.g., agarose), polyacrylamides, polystyrene, polyvinyl alcohol and silicones. In certain embodiments, depending on the context, the solid support can comprise tubes, plates, the wells of an assay or microtiter plate; such as those made from polystyrene or polyvinylchloride. In others it is a purification column (e.g., an affinity chromatography column). This term also includes a discontinuous solid support of discrete particles, such as those described in U.S. Pat. No. 4,275,149. Such materials are water insoluble and include cross-linked dextran (e.g., SEPHADEX™, Pharmacia Fine Chemicals, Piscataway, N.J.), agarose, polystyrene beads about 1 μm to about 5 mm in diameter, polyvinyl chloride, cross-linked polyacrylamide, nitrocellulose- or nylon-based webs such as sheets, strips or paddles)

The CCL3 epitope can be either covalently or non-covalently bound to the solid support by techniques such as covalent bonding via an amide or ester linkage or adsorption. After the CCL3 protein is affixed to the solid support, the solid support can be post-coated with a blocker (e.g., animal protein) to reduce non-specific adsorption of protein to the support surface.

The antibody containing biological samples can be either a crude sample or immunoglobulin purified samples (e.g., ammonium sulfate precipitated fraction and/or chromatography isolated).

The solid support is exposed to the biological sample so that the antibody, if any, is captured by the antigen. Typically, the antigen on the solid support will be present in excess so that the entire quantity of autoantibody may be bound. By then removing the solid support from the serum sample, the captured autoantibodies can be removed from non-specifically bound sample components.

Detection of immunocomplexes can be effected by adding labeled antibody-binding molecules such as staphylococcal protein A. The detectable label can be any one which is capable of producing, either directly or indirectly, a detectable signal. For example, the detectable label may be a radioisotope, a fluorescent or chemiluminescent compound, or a tag (such as described hereinabove and to which a labeled antibody can bind).

Thus, the label can be an enzyme such as horseradish peroxidase (HRP), glucose oxidase, alkaline phosphatase and the like. The selection of the enzyme will much depend on the source of the biological sample. For instance, high levels of peroxidase are present in blood cells and this may result in non-specific signal. However, a blocking treatment (e.g., with 0.3% solution of H₂O₂ in methanol for peroxidase) can be employed.

In cases where the major indicating group is an enzyme such as HRP or glucose oxidase, additional reagents are required to indicate that an immunocomplex has formed. Such additional reagents for HRP include hydrogen peroxide and an oxidation dye precursor such as diaminobenzidine. An additional reagent useful with glucose oxidase is 2,2,-azino-di-(3-ethyl-benzthiazoline-G-sulfonic acid) (ABTS).

Radioactive labels may also be used in accordance with the present invention. An exemplary radiolabeling agent is a radioactive element that produces γ ray emissions, such as ¹²⁵I. Methods of protein labeling are well-known in the art and described in details by Galfre et al., Meth. Enzyol., 73:3-46 (1981). The techniques of protein conjugation or coupling through activated functional groups are also applicable. See, for example, Aurameas et al., Scand. J. Immunol., 8(7):7-23 (1978); Rodwell et al., Biotech., 3:889-894 (1984); and U.S. Pat. No. 4,493,795. Peptides labeled according to the above described procedures can also be used for in vivo detection which as mentioned above, is also contemplated by the present invention.

The anti-CCL3 antibodies may be detected by any known assay method, such as competitive binding assays, direct and indirect sandwich assays, and immunoprecipitation assays [Zola, Monoclonal Antibodies: A Manual of Techniques, pp. 147-158 (CRC Press, Inc., 1987)].

Particularly preferred, are sensitive enzyme-linked immunosorbent assay (ELISA) methods which are described hereinabove, and in detail in U.S. Pat. Nos. 3,791,932; 3,839,153; 3,850,752; 3,879,262; and 4,034,074. Such ELISA assays can provide measurement of very low titers of the autoantibodies.

Another typical embodiment comprises radioimmunoassays (RIA) which are performed using a solid support which has been prepared as described above. The solid support is exposed to the biological sample in the presence of radiolabeled autoantibodies which can compete for binding to the immobilized antigen. In this way, the amount of radiolabel bound to the solid phase will be inversely proportional to the amount of autoantibodies initially present in the biological sample. After separation of the solid support, non-specifically bound radiolabel can be removed by washing, and the amount of radiolabel bound to the solid support determined. The amount of bound radiolabel, in turn, can be related to the amount of autoantibodies initially present in the sample.

As is shown in Example 2 in the Examples section that follows, anti-CCL3 autoantibodies were found in serum of prediabetic subjects. It has been found that the presence of autoantibodies to CCL3 is overlapping with the presence of autoantibodies to other autoantigens present in prediabetic patient sera. Hence, the present invention also contemplates determining of a presence or level of anti-CCL3 antibodies (e.g., at least one of autoantibodies against GAD, ICA and insulin) in combination with the teachings of the present invention, to improve accuracy of diagnosis.

Thus, according to one embodiment, determining the presence or level of anti-CCL 3 as described above further comprises with determining a presence or level of an autoantibody specific to a T1DM disease state. Particularly, according to another embodiment, the detection may be to autoantibodies specific for an antigen from glutamic acid decarboxylase (GAD), islet cell antigen (ICA) 512/IA-2 and insulin.

Thus, according to another embodiment of the present invention, determining a presence or a level of anti CCL3 antibodies is effected prior to the elicitation of an autoantibody specific to a T1DM disease state.

Preferably, ICAs are measured by indirect immunofluorescence using cryosections of human blood group 0 donor pancreata [as described in Vandewalle C L et al Diabetologia 36:1155-1162, (1993)]. Anti-Insulin IAAs, GADAs, and IA-2As are determined by liquid-phase radiobinding assays using, respectively, ¹²⁵I-labeled insulin, ³⁵S-labeled GAD65, and the ³⁵S-labeled intracellular domain of IA-2 (IA-21c) as tracer [Decochez K, et al., Diabetes Care 23:838-844, (2000)].

It will be appreciated that other autoantibodies specific to a T1DM disease state can be detected together with anti-CCL3. These include, but are not limited to the autoantibodies against the epitopes listed, together with the most common used autoantibodies, in the table below. Other autoantibodies and methods and compositions suitable for detecting them in patient sera are described in detail in Table 1 below, and in Devendra et al., Brit Med J 328(7442):750-754 (2004)].

TABLE 1

| | Sensitivity | Comment | reference |
|---|---|---|---|
| Insulin | 49-92% | Higher levels young | Palmer, J. P. et al (1983) |
| | | children | Science 222: 1337-1339 |
| GAD (Glutamic acid | 70-80% | Higher sensitivity | Clive et al., Autoimmunity |
| Decarboxylase) | | adult onset type 1A | reviews, 5: 424-428, 2006; WO |
| | | | 92/04632 |
| GAD 38 | 17% | Present in prediabetics, in | U.S. Pat. No. 6,960,448 |
| | | anti-GAD negantive | |
| | | subjects. | |
| ICA512/IA-2 | 74% | Tyrosine Phosphatase like | Rabin et al., Diabetes. |
| | | molecule | Feb; 41(2): 183-6 (1992). |
| IA-2β/Phogrin | 61% | Tyrosine Phosphatase like | Doi et al., PNAS 24; 103(4): |
| | | molecule | 885-890 (2006) |
| eCarboxypeptidase H | 10% | Infrequent | Castano, L. et al. J. Clin. |
| | | | Endocr. Metab. 73: 1197- |
| | | | 1201(1991) |
| GLIMA38 (glycated islet | 14-38% | Low diagnosis sensitivity | Winnock et al., Diabetes Care, |
| cell membrane- | | | 24(7): 1181-6(2001) |
| associated) | | | |
| GM2-1 | ? | Ganglioside glycolipid | Dotta, F., et al. (1992) |
| | | | Endocrinology 130: 37-42 |
| GT3 | ? | Glycolipid | Gillard, B. K., et al. (1989) |
| | | | Journal Immunol. Methods |
| | | | 142: 3826-3832 |
| PM-1 (69 kD ICA | ? | | U.S. Pat. No. 6,930,181 |
| protein) | | | |
| ICA69 | ? | Western blot assay with | Gaedigk R, et al., Genomics |
| | | poor specificity | 1996; 38: 382-391 |
| ICA12/SOX13 | <20% | Diabetes relatedness. | Park Y, et al., Ann. N.Y. Acad. |
| | | Does not usefully | Sci. 1005: 253-258 (2003); |
| | | distinguish T1DM | Bruce, S et al., Diab 20(3)198 (2003) |
| | | fromT2DM | |

The method of the present invention can be used to monitor anti diabetic treatment in a subject in need thereof.

This may be effected by exposing the subject to an anti-diabetic treatment; and determining a presence and/or a level of anti-CCL3 antibodies in a biological sample of the subject (as described above), wherein an alteration in a level of the anti-CCL3 antibodies in the biological sample following the anti diabetic treatment is indicative of treatment efficacy.

Determining anti-CCL3 antibodies can be effected following, as well as optionally prior to the antidiabetic treatment, in order to assess the influence of the treatment.

Examples of antidiabetic treatments which can be used in accordance with this aspect of the present invention include, but are not limited to insulin administration such as by injections (with e.g., a syringe or high air pressure jet injector), infusion, inhalation [e.g., Bellary and Brnett, Diab Vasc Dis Res. December; 3(3):179-85, 2006) or by an external insulin pump, as described in Diabetes Forecast (58(1):RG16, RG19-22, RG24-6, 2005]. Other routes for insulin administration include e.g., pills resistant to digestion, skin patch, intranasal or buccal spray [as further described in Lassmann-Vague and Raccah, Diabetes Metab. 32:513-22, (2006)]. In addition, in some cases, biguanides (e.g., Metformin), which are given to treat T2DM are useful in treating T1DM.

In severe cases (e.g., involving kidney failure or no response to insulin), T1DM subjects are treated by pancreas transplantation. Transplantation can be simultaneously done with a kidney transplant in order to increase the organ survival rate. Alternatively, transplantation can be done following a kidney transplantation or of the pancreas alone, according to the subjects specific condition and the physicians recommendation [further described in Morris et al., S D J. Med. 57(7):269-72, (2004)]. Other alternative transplantation strategies can be of islet cells, rather than a whole organ [Bertuzzi et al., Curr Mol. Med. June; 6(4):369-74, 2006] or cultured pancreas/beta cells [further described in Vinik et al., MedGenMed. 6:12, 2004].

Non conventional therapy for treating diabetes include, but are not limited to, acupuncture, biofeedback, and administration of chromium, ginseng, magnesium, and vanadium.

In addition, and together with the treatments described above, T1DM can be treated by change of dietary regimen in order to balance sugar intake and change of exercise regimen in order to control glucose levels. This, together with monitoring blood glucose levels serve to lower the effects of T1DM Any of the above mentioned reagents may be included in a kit.

Thus, according to another aspect of the present invention there is provided a kit for diagnosing T1DM the kit comprising a packaging material and at least one reagent for determining anti-CCL3 antibodies in a biological sample of the subject.

Thus, for example, antibodies and/or chemicals can be packaged in one or more containers with appropriate buffers and preservatives and used for diagnosis.

Preferably, the containers include a label. Suitable containers include, for example, bottles, vials, syringes, and test tubes. The containers may be formed from a variety of materials such as glass or plastic.

In addition, other additives such as stabilizers, buffers, blockers and the like may also be added.

The kit can also include instructions for determining if the tested subject is suffering from, or is at risk of T1DM.

Thus, a kit for screening blood, for example, can include the following components preferably in separate containers:

(a) a solid phase support coated with CCL3 epitope peptide.

(b) a diluent for the serum or plasma sample, e.g., normal goat serum or plasma;

(c) a labeled anti-(human IgG) antibody, e.g., goat anti-(human IgG) antibody in buffered, aqueous solution containing about 1% goat serum or plasma;

(d), a positive control, e.g., serum containing antibody against CCL3 protein; and/or

(e) a negative control, e.g., serum which does not contain antibody against CCL3 protein.

If the label is an enzyme, an additional component of the kit can be the substrate for the enzyme.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Detection of Anti CCL3 Autoantibodies in Sera of T1DM Subjects Immediately following Diagnosis

Autoantibody titers were determined for inflammatory mediators in subjects with early diagnosed T1DM. High autoantibody titer for CCL3 was shown.

Materials and Experimental Procedures

Experimental design—Autoantibody response was determined in 10 subjects diagnosed with T1DM (Type I Diabetes Center, Rambam, Haifa, Israel). Specifically, autoantibodies for the followinig chemokines were analysed: CCL2 (MCP-1), CCL3 (MIP-1α) and CCL4 (MIP-1β). The foregoing were found to be expressed in the inflamed pancreas during T1DM in mouse experimental models [Cameron M J et al., J Immunol 165:1102 (2000)]. In addition autoantibodies for the chemokine IL-8 (CXCL8) that is exclusively expressed in human, and is likely to be associated with inflammatory autoimmune diseases [Palacios, I et al., Clin Exp Immunol 111:588 (1998)] were analysed. The autoantibody titer of additional mediators that are likely to be associated with inflammatory autoimmune diseases was examined as well: the chemokines RANTES (CCL5), MIG (CXCL9), ITAC(CXCL11), and IP-10 (CXCL10); inflammatory cytokines IL-15 and IL-1β and the TNF family members CD40L, FASL and TRAIL

Detection of autoantibodies—ELISA plates (NUNC, Rofkilbe, Denmark) were coated with 10 ng/well of human CCL3 (R & D, Minneapolis, USA) and incubated with 200 μl 1% BSA/PBS blocking buffer for 1 hour at room temperature. Sera samples were subjected to sequential (×2) dilutions with the above blocking buffer and added to the ELISA plates (100 μl/well) for overnight incubation and washed 4 times with PBS/Tween 20 (0.05%). Thereafter, 50 μl goat anti-hIgG-HRP (Jackson, Pa., USA) was added (according the manufacturer protocol) in 1% BSA/PBS for 1 h and washed 4× with PBS/Tween 20 (0.05%). Substrate solution (TMB, DAKO, CA, USA) was then added at 50 μL per well. Reaction was terminated with the appearance of blue color, by adding 50 μl H₂SO₄ (1M). OD was determined at 450 nm with reference filter set to 630 nm.

### Results

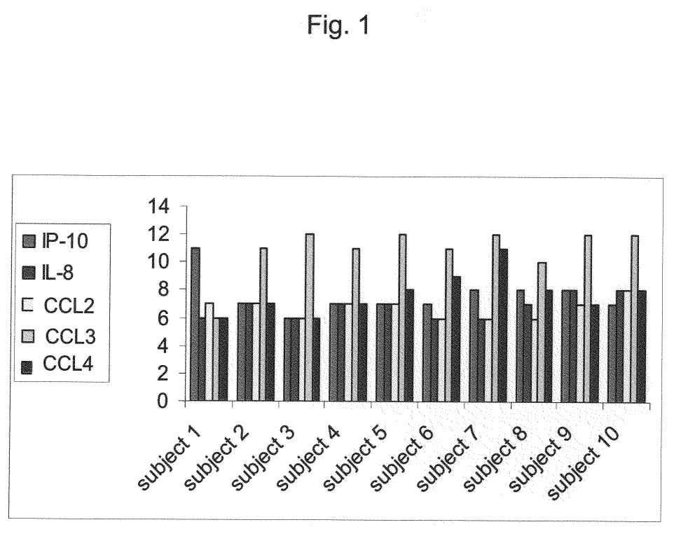

A higher autoantibody titer was found for CCL3 in early diagnosed T1DM subjects—As shown in FIG. 1, immediately following diagnosis (within one week of diagnosis), 9 out of 10 early-diagnosed subjects with T1DM developed a selective autoantibody response to CCL3, while no autoantibody response was found for any of other inflammatory mediators. Control subjects did not develop a notable antibody titer to any of the mediators, including CCL3.

### Example 2

### Detection of Positive CCL3 Autoantibody Response in Prolonged and Early Diagnosed T1DM Subjects

Materials and Experimental Procedures

Experimental design—The presence of anti CCL3 autoantibodies in blood samples was determined in four different groups: newly diagnosed T1DM subjects (within one week of diagnosis; n=30); prolonged T1DM (>5 years since diagnosis, n=38), healthy subjects (control; n=20) and prediabetic subjects (first degree relatives of subjects with T1DM with positive autoantibodies; n=20). For every patient, the titer for Type 1 Diabetes-related autoantibodies; anti insulin (CIAA), ICA and GAD was also determined.

Detection of autoantibodies—Detection of autoantibodies was effected as described in Example 1

Results

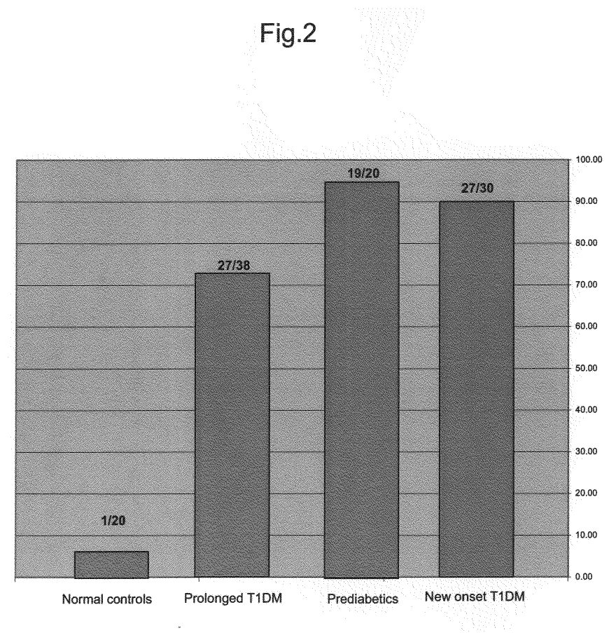

Autoantibody response to CCL3 in early diagnosed and prolonged prediabetic subjects—As shown in FIG. 2, 90% (27/30) of subjects who were only just diagnosed with T1DM were found to have a positive autoimmune response to CCL3. A high percentage of positive immune response was demonstrated by prediabetic subjects and prolonged T1DM subjects as well, where 19/20 (95%) and 27/38 (71%) displayed an autoantibody response, respectively. As can be seen in FIG. 2, positive autoantibody titer for CCL3 was exclusive for T1DM subjects as only 1/20 (5%) of healthy controls were positive for this antibody.

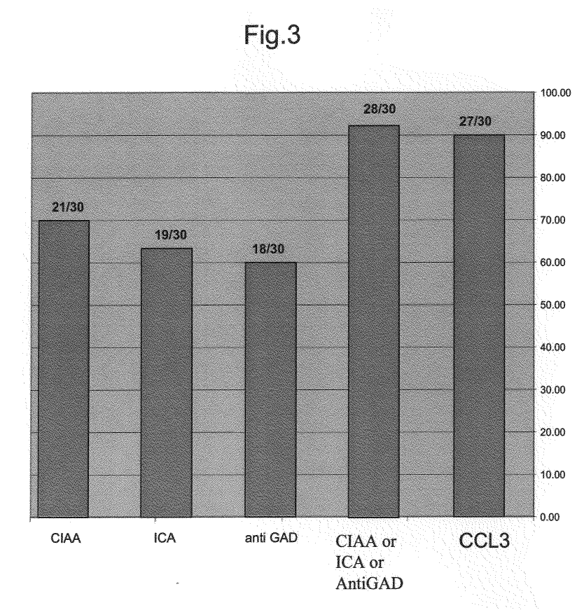

Autoantibody response to CCL3 as compared to other T1DM related autoantibodies—When compared to the conventional diagnostic tools for determining T1DM, results show, as depicted in FIG. 3, that CCL3 autoantibodies were positive in 90% of subjects (27/30), while the other, Type 1 Diabetes-related autoantibodies; anti insulin (CIAA), anti GAD and ICA antibodies were positive in 70%, 60% and 63% of subjects respectively (in 28 out of 30 subjects at least one of the 3 Diabetes-related autoantibodies were detected). Thus autoantibodies for CCL3 is a single diagnostic tool which is far more sensitive than the conventional tools, and as sensitive as the three conventional tools put together

Taken together, the present results substantiate that positive autoantibody titer for CCL3 is a single highly sensitive biomarker that can replace the 3 conventional less sensitive diagnostic tools used today. The antibody titer to CCL3 continues to be positive several years after the diagnosis of diabetes which further substantiates the marker found in the present invention as a new and effective diagnostic tool for the much needed purpose of distinguishing between adult Type 1 Diabetes Milletus (including LADA) and Type 2 DM patients.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. A method of diagnosing Type 1 Diabetes Mellitus (T1 DM) in a subject in need thereof, the method comprising determining a presence and/or a level of anti-CCL3 antibodies in a biological sample of the subject, wherein said presence or level above a predetermined threshold is indicative of T1DM, thereby diagnosing T1DM in the subject.

2. A kit for diagnosing T1DM, the kit comprising a packaging material and at least one reagent for determining anti-CCL3 antibodies in a biological sample of the subject.

3. A method of monitoring anti diabetic treatment in a subject in need thereof, the method comprising:
(a) exposing the subject to an anti-diabetic treatment; and
(b) determining a presence and/or a level of anti-CCL3 antibodies in a biological sample of the subject, wherein an alteration in a level of said anti-CCL3 antibodies in the biological sample following step (a) is indicative of treatment efficacy.

4. The method of claim 3, wherein step (b) is effected following and optionally prior to step (a).

5. The method of claim 3, wherein said anti-diabetic treatment comprises a drug selected from the group consisting of insulin, glucagon, glucose, biguanide, chromium, ginseng, magnesium and vanadium.

6. The method of claim 3, wherein said anti diabetic treatment is selected from the group consisting of pancreas transplantation, islet cell transplantation, and a life-style regimen.

7. The method of claim 1, wherein said determining a presence or a level of anti CCL3 antibodies is effected ex vivo.

8. The method of claim 1, wherein the T1DM comprises Latent Autoimmune Diabetes in Adults (LADA).

9. The method of claim 1, wherein said determining a presence or a level of anti CCL3 antibodies is effected prior to the elicitation of an autoantibody specific to a T1DM disease state.

10. The method of claim 1, further comprising determining a presence or a level of an autoantibody specific to a T1DM disease state.

11. The kit of claim 2, further comprising at least one reagent for determining a presence or level of an autoantibody specific to a T1 DM disease state.

12. The method of claim 10, wherein said autoantibody specific for a T1DM disease state is specific for an antigen selected from the group consisting of glutamic acid decarboxylase (GAD), islet cell antigen (ICA) 512/IA-2 and insulin.

13. The method of claim 1, wherein said threshold comprises an anti-CCL3 titer of log₂Ab 10-15.

14. The kit of claim 2, further comprising instructions as follows: wherein an anti-CCL3 titer of log₂Ab 10-15 is indicative of T1 DM.

15. The method of claim 1, wherein said determining is effected by ELISA.

16. The method of claim 3, wherein said determining a presence or a level of anti CCL3 antibodies is effected ex vivo.

17. The method of claim 3, wherein the T1DM comprises Latent Autoimmune Diabetes in Adults (LADA).

18. The method of claim 3, wherein said determining a presence or a level of anti CCL3 antibodies is effected prior to the elicitation of an autoantibody specific to a T1DM disease state.

19. The method of claim 3, further comprising determining a presence or a level of an autoantibody specific to a T1 DM disease state.

20. The method of claim 19, wherein said autoantibody specific for a T1DM disease state is specific for an antigen selected from the group consisting of glutamic acid decarboxylase (GAD), islet cell antigen (ICA) 512/IA-2 and insulin.
